(19) United States
(12) Patent Application Publication
(10) Pub. No.: US 2016/0303001 A1
(43) Pub. Date: Oct. 20, 2016

(54) POWDERY COSMETIC COMPOSITION

(71) Applicant: L'OREAL, Paris (FR)

(72) Inventor: Kazuyoshi TSUBATA, Yokohama-shi, Kanagawa (JP)

(21) Appl. No.: 15/101,575

(22) PCT Filed: Oct. 29, 2014
(86) PCT No.: PCT/JP2014/079380

§ 371 Date: Jun. 3, 2016

(30) Foreign Application Priority Data
Dec. 6, 2013 (JP) 2013-252904

(51) Int. Cl. A61K 8/02; A61Q 17/04; A61K 8/29; A61K 8/27; A61K 8/34; A61Q 1/12; A61K 8/37; A61K 8/73; A61K 8/891; A61K 8/26; A61K 8/19

(52) U.S. Cl. CPC A61K 8/022; A61K 8/26; A61Q 17/04; A61K 8/29; A61K 8/27; A61K 8/19; A61Q 1/12; A61K 8/37; A61K 8/732; A61K 8/891; A61K 8/345; A61K 2800/5922; A61K 2800/413

(57) ABSTRACT

The present invention relates to a powdery cosmetic composition comprising a pulverulent phase. The pulverulent phase comprises (i) perlite in an amount of from 5 wt % to 70 wt % in relation to the total weight of the composition, and (ii) at least one inorganic UV filter powder having an average primary particle size of lower than 200 nm. The powdery cosmetic composition according to the present invention can provide long-lasting cosmetic effects as well as good UV protecting effects.

### TECHNICAL FIELD

The present invention relates to a powdery cosmetic composition, a cosmetic process using the same, and a manufacturing process of the same.

### BACKGROUND ART

Long lastingness and good UV protecting effects are among the key functions of face make-up products in Asia, especially in hot and humid weather such as Indonesia, Thailand, etc.

To date, various documents regarding compositions including perlite have been published.

WO2012/035512 discloses a solid cosmetic makeup and/or care composition in the form of a powder comprising, in a physiologically acceptable medium, at least:
a fatty phase containing at least one silicone polyamide and a silicone resin, and
at least one pulverulent phase containing at least perlite.

WO2013/041274 discloses a solid cosmetic composition in the form of a powder, which is preferably compacted, comprising at least:
a pulverulent phase in an amount of greater than or equal to 35% by weight relative to the total weight of the composition, comprising at least one perlite in the form of particles in an amount of greater than or equal to 20% by weight relative to the total weight of the composition, and
a liquid fatty phase,
   in which the perlite particles and the liquid fatty phase are present in the composition in a respective total content such that the weight ratio of the perlite particles to the liquid fatty phase ranges from 2 to 25.

WO2012/035512 and WO2013/041274 disclose a solid cosmetic composition in the form of a powder, and they disclose incorporating perlite into a solid cosmetic composition especially in a powder form.

WO2009/007248 discloses pigments, comprising a plate-like substrate of perlite, and (a) a dielectric material, especially a metal oxide, having a high index of refraction; and/or (a) a metal layer, especially a thin semi-transparent metal layer; a process for their production and their use in paints, ink-j et printing, for dyeing textiles, for pigmenting coatings, printing inks, plastics, cosmetics, glazes for ceramics and glass.

WO2009/007248 discloses a pigment in which a metal oxide layer (e.g., TiO₂) is coated on a perlite base.

However, WO2012/035512 and WO2013/041274 are silent on using perlite with an inorganic UV filter powder at the same time. Also, WO2009/007248 does not disclose a specific formula for a powdery cosmetic composition.

### DISCLOSURE OF INVENTION

An objective of the present invention is to provide a powdery cosmetic composition which can produce long lasting cosmetic effects, as well as good UV filtering effects.

The above objective of the present invention can be achieved by a powdery cosmetic composition comprising a pulverulent phase, wherein the pulverulent phase comprises:

(i) perlite in an amount of from 5 wt % to 70 wt % in relation to the total weight of the composition, and.

(ii) at least one inorganic UV filter powder having an average primary particle size of lower than 200 nm, preferably from 5 nm to 150 nm, and more preferably from 10 nm to 100 nm.

The present invention also relates to a cosmetic process including a step of applying the powdery cosmetic composition according to the present invention to the skin, in particular the face.

The present invention also relates to a manufacturing process of a powdery cosmetic composition comprising a step of (i) mixing perlite and an inorganic UV filter powder having an average primary particle size of lower than 200 nm, preferably from 5 nm to 150 nm, and more preferably from 10 nm to 100 nm, to provide a pulverulent mixture, wherein an amount of the perlite is from 5 wt % to 70 wt % in relation to the total weight of the composition.

### BEST MODE FOR CARRYING OUT THE INVENTION

After diligent research, the inventors have discovered that a combination of perlite and inorganic UV filter powder can provide a cosmetic composition which can produce long lasting cosmetic effects, as well as better UV filtering effects.

### [Composition]

Thus, the present invention relates to a powdery cosmetic composition comprising a pulverulent phase, wherein the pulverulent phase comprises:

(i) perlite in an amount of from 5 wt % to 70 wt % in relation to the total weight of the composition, and.

(ii) at least one inorganic UV filter powder having an average primary particle size of lower than 200 nm, preferably from 5 nm to 150 nm, and more preferably from 10 nm to 100 nm.

The powdery cosmetic composition according to the present invention can provide long lasting cosmetic effects. Therefore, the powdery cosmetic composition can provide the skin with an attractive appearance for a long time. In addition, the powdery cosmetic composition according to the present invention can provide better UV protecting effects. Therefore, the powdery cosmetic composition exhibits a good performance in protecting the skin from UV stresses.

Furthermore, the powdery cosmetic composition according to the present invention does not require a large amount of inorganic UV filter powder to achieve sufficient UV protecting effects. Therefore, the powdery cosmetic composition can contribute to cost reduction, because the composition can be manufactured without any special industrial processes, for example special mixing or milling processes, which are expensive and complicated and generally applied in the case that a large amount of inorganic UV filter powder is included in the composition.

Hereinafter, the powdery cosmetic composition according to the present invention will be explained in a more detailed manner.

### (I) Pulverulent Phase

The powdery cosmetic composition according to the present invention comprises a pulverulent phase. The pulverulent phase comprises perlite and at least one inorganic UV filter powder. The pulverulent phase is a solid state at room temperature (25° C.) under atmospheric pressure (760 mmHg).

The powdery composition according to the present invention advantageously has a pulverulent phase content of 40% by weight or more, preferably 50% by weight or more, more preferably from 60% to 98% by weight, and still more preferably from 70% to 95% by weight in relation to the total weight of the powdery cosmetic composition.

Perlite

Perlite is used for providing the powdery cosmetic composition according to the present invention with long lasting cosmetic effects. Furthermore, together with the inorganic UV filter powder, the perlite can produce synergy effects that enhance the UV protecting effects which are produced by the inorganic UV filter powder in the composition.

The perlite is preferably present in a fine particle form in the composition. More preferably, the perlite is present in the form of primary particles in the composition. The perlite in the primary particle form is also present in a free particle form. The term “free particle form” here means that the particle does not bind chemically or physically to other particles.

The average primary particle size of the perlite ranges from 0.5 μm to 50 μm, preferably 1 μm to 40 μm, and more preferably 3 μm to 30 μm. The average primary particle size here means a number-average size mean diameter which is given by the statistical particle size distribution to half of the population, referred to as D50. For example, such a number-average size mean diameter of the perlite can be measured by a laser diffraction particle size distribution analyzer, such as Mastersizer 2000 by Malvern Corp.

The perlite is generally obtained from natural glass of volcanic origin, of light-grey or glossy black colour, resulting from the rapid cooling of lava, and which is in the form of small particles resembling pearls. When heated above 800° C., perlite has the particular feature of losing the water it contains and of adopting a porous expanded form (representing from four to twenty times its initial volume), enabling it to absorb large amounts of liquid, in particular oil and water. In this form, perlite has a white colour and pore structure.

The perlite, which is of mineral origin, is directly extracted from the ground and then finely ground to obtain a very fine white powder: perlite powder or perlite particles.

The perlite particles are thus particles of amorphous mineral materials, which are advantageously expanded, derived from at least one volcanic rock.

These particles comprise at least two elements chosen from silicon, aluminium and magnesium.

More particularly, these mineral materials are obtained by thermal expansion of a volcanic or “effusive” rock comprising from 1% to 10% by weight of water and preferably 1% to 5% by weight of water and less than 10% by weight of crystalline rock relative to the total weight of the rock composition and preferably followed by grinding. The temperature of the expansion process may range from 700 to 1500° C. and preferably from 800 to 1100° C. The expansion process described in U.S. Pat. No. 5,002,698 may especially be used.

Volcanic or “effusive” rocks are generally produced by the rapid cooling of liquid magma in contact with air or water (a quenching phenomenon which yields a hyaline rock). The volcanic rocks that may be used according to the present invention are chosen from those defined according to the Streckeisen classification (1974). Among these volcanic rocks, mention may be made especially of trachytes, latites, andesites, basalts, rhyolites and dacites. Rhyolites and dacites are particularly suitable for use, and even more particularly rhyolites.

The perlite particles that may be used according to the invention are preferably aluminosilicates of volcanic origin. They may advantageously have the following composition:

70.0-75.0% by weight of silica SiO₂
12.0-15.0% by weight of oxide of aluminium oxide Al₂O₃
3.0-5.0% of sodium oxide Na₂O
3.0-5.0% of potassium oxide K₂O
0.5-2% of iron oxide Fe₂O₃
0.2-0.7% of magnesium oxide MgO
0.5-1.5% of calcium oxide CaO
0.05-0.15% of titanium oxide TiO₂

In the implementation of the present invention, the perlite undergoes a first milling step so as to form perlite particles, and is dried and then calibrated. The product obtained, known as perlite ore, is grey-coloured and has a size of about 100 μm. The perlite ore is then expanded (1000° C./2 seconds) to give more or less white particles. When the temperature reaches 850-900° C., the water trapped in the structure of the material evaporates and brings about the expansion of the material relative to its original volume. The expanded perlite particles in accordance with the invention may be obtained via the expansion process described in U.S. Pat. No. 5,002,698.

Preferably, the perlite particles used are then milled in a second milling step in order to further reduce the size of the perlite particles used; in this case, they are referred to as expanded milled perlite (EMP), to form fine particles.

Preferably, the perlite has a platelet shape, and consequently is usually called a lamellar filler, as opposed to a spherical filler which is of a globular shape.

The perlite advantageously has a coefficient of expansion of from 2 to 70.

Preferably, the perlite has an untamped density at 25° C. ranging from 10 to 400 kg/m³ (standard DIN 53468) and more preferably from 10 to 300 kg/m³.

According to one particular embodiment of the invention, the perlite has a silica content of greater than or equal to 65% by weight relative to the total weight of the composition of the material. According to one particular embodiment of the invention, the perlite has a spontaneous pH, measured at 25° C. in a dispersion in water at 10% by weight, ranging from 6 to 8.

Preferably, the expanded perlite used in the present invention has a water-absorbing capacity, measured at the wet point, ranging from 200% to 1500% and preferably from 250% to 800%.

The wet point corresponds to the amount of water that needs to be added to 1 g of particles in order to obtain a homogeneous paste. This method is derived directly from that of the oil uptake applied to solvents. The measurements are taken in the same manner by means of the wet point and the flow point, which have, respectively, the following definitions:

Wet Point: weight expressed in grams per 100 g of product corresponding to the production of a homogeneous paste during the addition of a solvent to a powder.

Flow Point: weight expressed in grams per 100 g of product at and above which the amount of solvent is greater than the capacity of the powder to retain it. This is reflected by the production of a more or less homogeneous mixture that flows over a glass plate.

The wet point and the flow point are measured according to the following protocol:

### Protocol for Measuring the Water Absorption

1) Equipment used
Glass plate (25×25 mm)
Spatula (wooden shaft and metal part, 15×2.7 mm)
Silk-bristled brush

### Balance

### 2) Procedure

The glass plate is placed on the balance and 1 g of perlite is weighed out. A beaker containing a solvent and a liquid sampling pipette is placed on the balance. The solvent is gradually added to the powder while regularly mixing (every 3 to 4 drops) with the spatula.

The weight of solvent needed to obtain the wet point is noted. Further solvent is added and the weight which makes it possible to reach the flow point is noted. The average over three tests is determined.

The perlite used according to the invention is especially commercially available from the company World Minerals under the trade name Perlite P1430, Perlite P2550, Perlite P2040 or OpTiMat™ 1430 OR or 2550 OR.

The perlite is present in a composition in accordance with the present invention in a content ranging from 5% to 70% by weight relative to the total weight of the composition, preferably from 7% to 68% by weight relative to the total weight of the composition, and more preferably from 10% to 65% by weight relative to the total weight of the composition.

The perlite and the pulverulent phase may be present in the composition in a respective total content such that the weight ratio of the perlite to the pulverulent phase ranges from 0.02 to 1, preferably from 0.05 to 1, and even more preferably from 0.1 to 1.

Inorganic UV Filter Powder

Inorganic UV filter powder is used for providing the powdery cosmetic composition according to the present invention with UV protecting effects.

The inorganic UV filter powder is preferably present in a fine particle form in the composition. More preferably, the inorganic UV filter powder is present in the form of primary particles in the composition. The inorganic UV filter powder in the primary particle form is also present in a free particle form. The term “free particle form” here means that the particle does not fuse chemically or physically to other particles (e.g., not coat other particles).

The term “UV” here comprises the UVB region (260-320 nm in wavelength) and the UVA region (320-400 nm in wavelength). Therefore, a UV filter means any material which has filtering effects in the wavelength of UV, in particular the UVA and UVB regions.

The inorganic UV filter powder used for the present invention may be active in the UV-A and/or UV-B region, preferably in the UV-B region or in the UV-A and UV-B region. The powdery cosmetic composition according to the present invention can comprise a further additional UV filter other than the inorganic UV filter powder. It is preferable that the active UV filtering region of the inorganic UV filter powder and that of the additional UV filter are complementary to each other, in order to provide comprehensive UV protection. For example, it is preferable that the inorganic UV filter powder is active at least in the UV-B region and the additional UV filter is active at least in the UV-A region. The inorganic UV filter powder may be hydrophilic and/or lipophilic.

The inorganic UV filter powder may be in the form of a fine particle having an average primary particle size of lower than 200 nm, preferably lower than 180 nm, and more preferably from 5 nm to 180 nm, and even more preferably from 5 nm to 150 nm, and still more preferably from 10 nm to 100 nm. The average primary particle size here means a number-average size mean diameter which is given by the statistical particle size distribution to half of the population, referred to as D50. For example, such a number-average size mean diameter of the inorganic UV filter powder can be measured by SEM (Scanning Electron Microscope) and/or TEM (Transmission Electron Microscope). In these measurements with SEM and/or TEM, generally, an equivalent circle diameter of each of measured particles is used as a diameter of each of the particles, and at least 80 particles are measured for determining the number-average size mean diameter. The equivalent circle diameter can be determined by image analysis using image analysis software, such as “WinRoof” from Mitani Syoji.

The inorganic UV filter powder may be selected from metal oxides, such as titanium oxide (amorphous or crystalline in the rutile and/or anatase form), zinc oxide, zirconium oxide or cerium oxide, which are all well-known UV photoprotective agents. Preferably, the inorganic UV filter powder is selected from the group consisting of titanium dioxide, zinc oxide, and cerium oxide.

The inorganic UV filter powder may or may not be coated. The inorganic UV filter powder may have at least one coating. The coating may comprise at least one compound selected from the group consisting of alumina, silica, aluminum hydroxide, silicones, silanes, fatty acids or salts thereof (such as sodium, potassium, zinc, iron or aluminum salts), fatty alcohols, lecithin, amino acids, polysaccharides, proteins, alkanolamines, waxes such as beeswax, (meth)acrylic polymers, organic UV filters, and (per)fluoro compounds.

It is preferable for the coating to include at least one organic UV filter powder. As the organic UV filter in the coating, a dibenzoylmethane derivative such as butyl methoxydibenzoylmethane (Avobenzone) and 2,2′-Methylenebis[6-(2H-Benzotriazol-2-yl)-4-(1,1,3,3-Tetramethyl-Butyl)Phenol](Methylene Bis-Benzotriazolyl Tetramethylbutylphenol) marketed as “TINOSORB M” by BASF may be preferable.

In a known manner, the silicones in the coating(s) may be organosilicon polymers or oligomers comprising a linear or cyclic and branched or crosslinked structure, of variable molecular weight, obtained by polymerization and/or polycondensation of suitable functional silanes and essentially composed of a repetition of main units in which the silicon atoms are connected to one another via oxygen atoms (siloxane bond), optionally substituted hydrocarbon radicals being connected directly to the said silicon atoms via a carbon atom.

The term “silicones” also encompasses silanes necessary for their preparation, in particular alkylsilanes.

The silicones used for the coating(s) can preferably be selected from the group consisting of alkylsilanes, polydialkylsiloxanes and polyalkylhydrosiloxanes. More preferably, the silicones are selected from the group consisting of octyltrimethylsilane, polydimethylsiloxanes and poly methylhydrosiloxanes.

Of course, the inorganic UV filter powder made of metal oxides may have been treated with other surfacing agents before treatment with silicones, in particular with cerium oxide, alumina, silica, aluminum compounds, silicon compounds or their mixtures.

The coated inorganic UV filter powder may be prepared by subjecting the inorganic UV filter powder to one or more surface treatments of a chemical, electronic, mechanochemical and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filter powder may be titanium oxides coated:

with silica, such as the product “Sunveil” from Ikeda;
with silica and with iron oxide, such as the product “Sunveil F” from Ikeda;
with silica and with alumina, such as the products “Microtitanium Dioxide MT 500 SA” from Tayca, “Tioveil” from Tioxide, and “Mirasun TiW 60” from Rhodia;
with alumina, such as the products “Tipaque TTO-55 (B)”, “Tipaque TTO-55 (A)” and “MPT-141” (primary particle size: 95-125 nm) from Ishihara Sangyo, and “UVT 14/4” from Kemira;
with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck;
with alumina and with aluminum laurate, such as the product “Microtitanium Dioxide MT 100 S” from Tayca;
with iron oxide and with iron stearate, such as the product “Microtitanium Dioxide MT 100 F” from Tayca;
with zinc oxide and with zinc stearate, such as the product “BR351” from Tayca;
with silica and with alumina and treated with a silicone, such as the products “Microtitanium Dioxide MT 600 SAS”, “Microtitanium Dioxide MT 500 SAS” and “Microtitanium Dioxide MT 100 SAS” from Tayca;
with silica, with alumina and with aluminum stearate and treated with a silicone, such as the product “STT-30-DS” from Titan Kogyo;
with silica and treated with a silicone, such as the product “UV-Titan X 195” from Kemira;
with alumina and treated with a silicone, such as the products “Tipaque TTO-55 (S)” from Ishihara Sangyo or “UV Titan M 262” from Kemira;
with triethanolamine, such as the product “STT-65-S” from Titan Kogyo;
with stearic acid, such as the product “Tipaque TTO-55 (C)” from Ishihara Sangyo;
with aluminum hydroxide and with stearic acid, such as the product “ST-455” and “ST-485SA15” from Titan Kogyo;
with aluminum hydroxide and with silica, such as the product “ST-495M” from Titan Kogyo; or
with sodium hexametaphosphate, such as the product “Microtitanium Dioxide MT 150 W” from Tayca.

Other titanium oxides treated with a silicone are preferably TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO₂Si₃” by Cardre, and anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

Preferably, the following coated TiO₂ can be used as the coated inorganic UV filter powder:

Alumina (and) TiO₂, such as the product “MPT-141” from Ishihara Sangyo, with a primary particle size of from 95 to 125 nm;
Stearic acid (and) Aluminum Hydroxide (and) TiO₂, such as the product “MT-100 TV” from Tayca, with a mean primary particle diameter of 15 nm;
Dimethicone (and) Stearic Acid (and) Aluminum Hydroxide (and) TiO₂, such as the product “SA-TTO-S4” from Miyoshi Kasei, with a mean primary particle diameter of 15 nm;
Silica (and) TiO₂, such as the product “MT-100 WP” from Tayca, with a mean primary particle diameter of 15 nm;
Dimethicone (and) Silica (and) Aluminum Hydroxide (and) TiO₂, such as the product “MT-Y02” and “MT-Y-110 M3S” from Tayca, with a mean primary particle diameter of 10 nm;
Dimethicone (and) Aluminum Hydroxide (and) TiO₂, such as the product “SA-TTO-S3” from Miyoshi Kasei, with a mean primary particle diameter of 15 nm;
Dimethicone (and) Alumina (and) TiO₂, such as the product “UV TITAN M170” from Sachtleben, with a mean primary particle diameter of 15 nm; and
Silica (and) Aluminum Hydroxide (and) Alginic Acid (and) TiO₂, such as the product “MT-100 AQ” from Tayca, with a mean primary particle diameter of 15 nm.

In terms of UV filtering ability, TiO₂ coated with at least one organic UV filter is more preferable. For example, Avobenzone (and) Stearic Acid (and) Aluminum Hydroxide (and) TiO₂, such as the product “HXMT-100ZA” from Tayca, with a mean primary particle diameter of 15 nm, can be used.

The uncoated titanium oxides are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxides are, for example:

those marketed under the trademark “Z-cote” by Sunsmart;
those marketed under the trademark “Nanox” by Elementis; and
those marketed under the trademark “Nanogard WCD 2025” by Nanophase Technologies.

The coated zinc oxides are, for example:

those marketed under the trademark “Oxide Zinc CS-5” by Toshiba (ZnO coated with polymethylhydrosiloxane);
those marketed under the trademark “Nanogard Zinc Oxide FN” by Nanophase Technologies (as a 40% dispersion in Finsolv TN, C₁₂-C₁₅ alkyl benzoate);
those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane);
those marketed under the trademark “NFD Ultrafine ZnO” by Daikin (ZnO coated with phosphate of perfluoroalkyl and a copolymer based on perfluoroalkylethyl as a dispersion in cyclopentasiloxane);
those marketed under the trademark “SPD-Z1” by Shin-Etsu (ZnO coated with a silicone-grafted acrylic polymer dispersed in cyclodimethylsiloxane);
those marketed under the trademark “Escalol Z100” by ISP (alumina-treated ZnO dispersed in an ethylhexyl methoxycinnamate/PVP-hexadecene copolymer/methicone mixture); and
those marketed under the trademark “Fuji ZnO-SMS-10” by Fuji Pigment (ZnO coated with silica and polymethylsilsesquioxane); those marketed under the trademark “Nanox Gel TN” by Elementis (ZnO dispersed at 55% in C₁₂-C₁₅ alkyl benzoate with hydroxystearic acid polycondensate).

The uncoated cerium oxide is marketed, for example, under the trademark “Colloidal Cerium Oxide” by Rhone-Poulenc.

Mention may also be made of mixtures of metal oxides, in particular of titanium dioxide and of cerium dioxide, including a mixture of equal weights of titanium dioxide coated with silica and of cerium dioxide coated with silica marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

The UV filter powder can generally be present in the composition according to the present invention in proportions ranging from 3% to 40% by weight, preferably ranging from 5% to 30% by weight, and more preferably 10% to 25% by weight, with respect to the total weight of the composition.

The UV protecting effects by the inorganic UV filter powder can be estimated by measuring the UV-ray transmittance thereof. Generally, such UV-ray transmittance can be measured with a UV/Vis spectrometer.

Additional Filler(s)

The pulverulent phase according to the present invention may preferably comprise at least one additional filler.

The term “fillers” should be understood as meaning colorless or white solid particles of any form, which are in a form that is insoluble and dispersed in the medium of the powdery cosmetic composition. Mineral or organic in nature, they make it possible to impart the powdery cosmetic powdery cosmetic composition with softness, mattness and uniformity of makeup. The fillers used in the compositions according to the present invention may be non-spherical fillers, in particular lamellar fillers, or spherical fillers (globular fillers). The fillers may also include fiber(s). The fillers according to the present invention may or may not be surface-coated.

### Non-Spherical Fillers

The “non-spherical” filler may be of any form other than spherical, for example, platelet-shaped, and oblong, irrespective of their crystallographic form (for example lamellar, cubic, hexagonal, and orthorhombic). Non-spherical fillers are preferably selected from non-spherical mineral fillers. Among the non-spherical mineral fillers that may be used in the powdery cosmetic compositions according to the present invention, mention may be made of talc, mica, silica, magnesium aluminium silicate, trimethyl siloxysilicate, kaolin, bentone, calcium carbonate, magnesium hydrogen carbonate, hydroxyapatite, boron nitride, fluorphlogopite, sericite, calcinated talc, calcinated mica, calcinated sericite, synthetic mica, lauroyl lysine, metal soap, bismuth oxychloride, barium sulfate, magnesium carbonate, and mixtures thereof.

According to the present invention, the non-spherical filler may have been surface-treated with a surface treatment agent comprising at least one silicone oil.

The silicone oil may be selected from polydialkylsiloxanes such as polydimethylsiloxane, polyalkylaryldiloxanes such as polymethylphenylsiloxane, polydiarylsiloxanes such as polydiphenylsiloxanes, polyalkylhydrogensiloxanes such as methylhydrogenpolysiloxane, and modified-polysiloxanes.

The modified-polysiloxanes may be chosen from the following formulae:
(a¹) modified polysiloxanes bearing polyethers, chosen from compounds of formula (III):

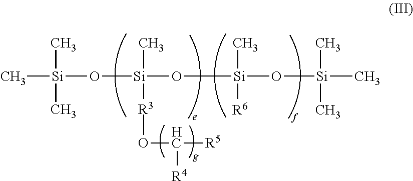

wherein
   R³ comprises —(CH₂)ₕ—;
   R⁴ comprises —(CH₂)ᵢ— CH₃;
   R⁵ is chosen from —OH, —COOH, —CH═CH₂, —C(CH₃)═CH₂ and —(CH₂)ⱼ— CH₃;
   R⁶ comprises —(CH₂)ₖ—CH₃;
   g and h independently range from 1 to 15;
   j and k independently range from 0 to 15;
   e ranges from 1 to 50; and
   f ranges from 1 to 300;
(a²) modified polysiloxanes bearing polyesters, chosen from compounds of formula (IV):

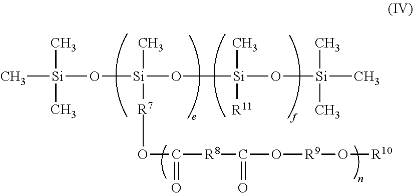

wherein
   R⁷, R⁸ and R⁹ are independently chosen from —(CH₂)_{q}—;
   R¹⁰ is chosen from —OH, —COOH, —CH═CH₂, —C(CH₃)═CH₂ and —(CH₂)ᵣ— CH₃;
   R¹¹ comprises —(CH₂)ₛ— CH₃;
   n and q independently range from 1 to 15;
   r and s independently range from 0 to 15;
   e ranges from 1 to 50; and
   f ranges from 1 to 300;
(a³) modified polysiloxanes bearing epoxy radicals, chosen from compounds of formula (V):

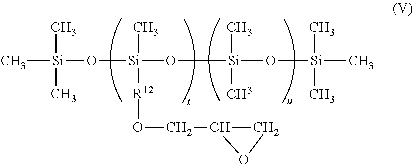

wherein
   R¹² comprises —(CH₂)ᵥ—;
   v ranges from 1 to 15;
   t ranges from 1 to 50; and
   u ranges from 1 to 300;
      and
mixtures thereof.

Alternatively, the modified-polysiloxane may be chosen from compounds of formula (VI):

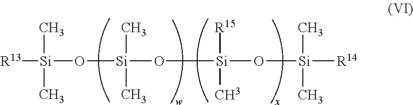

wherein
R¹³ and R¹⁴ are independently chosen from —OH, R¹⁶OH and R¹⁷COOH;
R¹⁵ is chosen from —CH₃ and —C₆H5;
R¹⁶ and R¹⁷ comprise —(CH₂)_{y}—;
y ranges from 1 to 15;
w ranges from 1 to 200; and
x ranges from 0 to 100.

It is preferable that the silicone oil is a polydialkylsiloxane such as polydimethylsiloxane or a mixture of polydialkylsiloxanes.

The surface treatment agent for the non-spherical filler may comprise at least one dimethylpolysiloxane.

According to one embodiment of the present invention, the surface treatment of the non-spherical filler may be chosen from the following treatments:

PEG-silicone treatments, for instance the AQ surface treatment sold by LCW;
methicone treatments, for instance the SI surface treatment sold by LCW; and
dimethicone treatments, for instance the Covasil 3.05 surface treatment sold by LCW, or the SA surface treatments sold by Miyoshi Kasei, and in particular the product SA-TA-13R sold by MIYOSHI KASEI (INCI Name Talc and dimethicone).

In a preferred embodiment, a dimethicone-treated talc can be used as the non-spherical filler.

According to the present invention, the non-spherical filler may have been surface-treated with a surface treatment agent comprising at least one amino acid and/or a derivative thereof.

The amino acid may preferably be selected from the group consisting of proline, hydroxyproline, alanine, glycine, sarcosine, aspartic acid, and glutamic acid.

The amino acids may be L-isomers or a mixture of L-isomers and D-isomers.

It is preferable that the non-spherical filler has been coated with:

(a) at least one selected from proline, hydroxyproline and derivatives thereof, and/or
(b) at least one selected from alanine, glycine, sarcosine and derivatives thereof; and/or
(c) at least one selected from aspartic acid, glutamic acid and derivatives thereof.

The derivatives of the amino acids may be selected from salts of the amino acids, and N-acylated amino acids and salts thereof.

It is preferable that two of the components (a) to (c) be used together, and it is more preferable that all of the components (a) to (c) be used together. If two or more of the components (a) to (c) are used, the type of the derivatives and/or salts may be the same or different.

The N-acyl group of the N-acylated amino acid may be a linear or branched, saturated or unsaturated acyl group with C₈-C₂₂ carbon atoms, preferably C₁₂-C₁₈ carbon atoms. It is preferable that the N-acyl group is a linear saturated acyl group, such as a palmitoyl group.

The salt of the amino acid or the N-acylated amino acid is not limited but may be in the form of a metal salt with a metal element such as Na, K, Ba, Zn, Ca, Mg, Fe, Zr, Co, Al, Ti and the like; an onium salt such as an ammonium salt; and a salt with an organic alkanolamine such as monoethanolamine, diethanolamine, triethanolamine, 2-amino-2-methylpropanol, 2-amino-2-methyl-1,3-propanediol, and triisopropanolamine. It is preferable that the salt is a metal salt with Na, K, Ca, Mg or Al.

It is more preferable that the non-spherical filler has been coated with a mixture (referred to as “lipo-amino acid composition”) of at least one fatty acid, such as a C₁₂-C₁₈ fatty acid, and/or a salt of the fatty acid, and

(a) at least one selected from proline, hydroxyproline and derivatives thereof; and/or
(b) at least one selected from alanine, glycine, sarcosine and derivatives thereof; and/or
(c) at least one selected from aspartic acid, glutamic acid and derivatives thereof.

As the fatty acid, a linear, branched or cyclic fatty acid, preferably C₁₂-C₁₈, can be used. A plurality of fatty acids may be used. As examples of the fatty acid, mention may be made of lauric acid, myristic acid, isomyristic acid, palmitic acid, isopalmitic acid, stearic acid, isostearic acid, oleic acid, myristoleic acid, elaidic acid, linoleic acid, and linolenic acid. As examples of the salt of the fatty acid, mention may be made of a metal salt with a metal element such as Na, K, Ba, Zn, Ca, Mg, Fe, Zr, Co, Al, Ti or the like. Lauric acid, myristic acid, palmitic acid and stearic acid as well as a metal salt thereof with Na, K, Ca, Al or Mg are preferable. Lauric acid, myristic acid and palmitic acid are more preferable. Palmitic acid is most preferable.

In the lipo-amino acid composition, each of the fatty acid (or a salt thereof) and any of the components (a) to (c) may represent 0.5% by weight or more, preferably 5% by weight or more, and more preferably 10% by weight or more, relative to the total weight of the lipo-amino acid composition.

It is most preferable that the lipo-amino acid composition comprises all of the components (a) to (c) as well as at least one fatty acid, such as a C₁₂-C₁₈ fatty acid, and/or a salt of the fatty acid.

For example, a mixture of palmitic acid, palmitoyl proline, palmitoyl sarcosinate, and palmitoyl glutamate can be used as the lipo-amino acid composition. A mixture of palmitic acid, palmitoyl proline, sodium palmitoyl sarcosinate, and magnesium palmitoyl glutamate is more preferable.

In the lipo-amino acid composition comprising all of the components (a) to (c), each of the fatty acid (or a salt thereof) and any of the components (a) to (c) may represent 0.5% by weight or more, preferably 5% by weight or more, and more preferably 10% by weight or more, relative to the total weight of the lipo-amino acid composition. It is possible that the lipo-amino acid composition comprises 5-50% by weight of the component (a), 5-50% by weight of the component (b), 5-25% by weight of the component (c) and 5-50% by weight of the fatty acid (or a salt thereof), relative to the total weight of the lipo-amino acid composition.

The lipo-amino acid composition can be prepared by a known method. For example, it is possible to prepare the lipo-amino acid composition in accordance with the methods described in WO 98/09611, WO 99/04757, JP-A-2000-191426 and the like. The above lipo-amino acid composition is also marketed in the name of Sepifeel One sold by Seppic in France.

The surface-treated non-spherical filler can be prepared by coating the filler with any of the components (a) to (c), a mixture of two or more of the components (a) to (c), or the lipo-amino acid composition described above.

The coating can be performed by a known method. For example, the non-spherical filler can be added into a solution of any of the components (a) to (c), a mixture of two or more of the components (a) to (c), or the lipo-amino acid composition described above; the filler is dispersed in the solution; and the dispersion is filtered, washed and dried. The solvent of the solution may be selected from water, aqueous solvents such as methanol and ethanol, and non-aqueous solvents such as ethyl acetate, depending on the nature of the components (a) to (c) and the like.

The amount of the coating depends on the type of the filler, and can be 0.1 to 30% by weight, preferably 1.0 to 10% by weight, relative to the total weight of the filler.

The filler may preferably be pre-coated with at least one oxide or hydroxide of a metal element such as aluminum, calcium, magnesium, cerium, silicon, zirconium, titanium, zinc, iron, cobalt, manganese, nickel, and tin. Aluminum hydroxide is more preferable. Further, the filler may preferably be pre-coated with a silicone compound, a fatty acid, a metal soap, a fluorine-based compound, a silane-coupling agent, and the like.

In one embodiment, the non-spherical filler coated with the lipo-amino acid composition comprising at least one fatty acid, such as a C₁₂-C₁₈ fatty acid, and/or a salt of the fatty acid, and the components (a) to (c) is/are commercially available.

For example, mica coated with palmitoyl proline, sodium palmitoyl sarcosinate, magnesium palmitoyl glutamate or palmitic acid has been marketed by Miyoshi Kasei Inc. in Japan.

In another embodiment, non-spherical fillers which have been surface-treated as follows are commercially available:
a PEG-silicone treatment, for instance the AQ surface treatment sold by LCW;
a lauroyllysine treatment, for instance the LL surface treatment sold by LCW;
a lauroyllysine dimethicone treatment, for instance the LL/SI surface treatment sold by LCW;
a disodium stearoyl glutamate treatment, for instance the NAI surface treatment sold by Miyoshi;
a dimethicone/disodium stearoyl glutamate treatment, for instance the SA/NAI surface treatment sold by Miyoshi;
a microcrystalline cellulose and carboxymethylcellulose treatment, for instance the AC surface treatment sold by Daito;
an acrylate copolymer treatment, for instance the APD surface treatment sold by Daito;
a sodium dilauramidoglutamide lysine treatment, for instance the ASL treatment sold by Daito; and
a sodium dilauramidoglutamide lysine/isopropyl titanium triisostearate treatment, for instance the ASL treatment sold by Daito.

### Spherical Fillers

Among spherical fillers that may be used, mention may be made of spherical mineral fillers and spherical organic fillers. By “spherical fillers,” one must understand the fillers or particles comprising at least one generally rounded portion, preferably defining at least a portion of a sphere, possibly internally defining a concavity or depression.

### (Spherical Mineral Fillers)

Among spherical mineral fillers that may be used, mention may be made of silica microspheres, for example, of open porosity, such as hollow silica microspheres, including the products “Silica Beads SP 700/HA(R)” and “Silica Beads SB 700(R)” from Maprecos, glass or ceramic microcapsules, silica-based fillers, for instance Aerosil 200 or Aerosil 300; Sunsphere H-33 and Sunsphere H-51 sold by Asahi Glass; Chemicelen sold by Asahi Chemical; and composites of silica and of titanium dioxide, for instance the TSG series sold by Nippon Sheet Glass.

### (Spherical Organic Fillers)

Among spherical organic fillers that may be used, mention may be made of (meth)acrylic or (meth)acrylate powders, for example, polymethylmethacrylate powders; polyacrylonitrile powders; organopolysiloxane powders, polyamide powders (Nylon® Orgasol from Atochem), poly-β-alanine powders and polyethylene powders, polytetrafluoroethylene powders (Teflon®), lauroyllysine, starch, tetrafluoroethylene polymer powders, hollow polymer microspheres, for example comprising an (alkyl)acrylate, such as Expancel® (Nobel Industrie), metal soaps derived from organic carboxylic acids containing from 8 to 22 carbon atoms and preferably from 12 to 18 carbon atoms, for example zinc stearate, magnesium stearate, lithium stearate, zinc laurate, magnesium myristate, Polypore® L200 (Chemdal Corporation), silicone resin microbeads (for example Tospearl® from Toshiba), polyurethane powders, in particular powders of crosslinked polyurethane comprising a copolymer, the said copolymer comprising trimethylol hexyl lactone, for instance the hexamethylene diisocyanate/trimethylol hexyl lactone polymer sold under the name Plastic Powder D-400® or Plastic Powder D-800® by the company Toshiki, carnauba microwaxes, such as the product sold under the name Micro Care 350® by the company Micro Powders, synthetic microwaxes, such as the product sold under the name MicroEase 114S® by the company Micro Powders, microwaxes formed from a mixture of carnauba wax and polyethylene wax, such as those sold under the name of Micro Care 300® and 310® by the company Micro Powders, microwaxes formed from a mixture of carnauba wax and of synthetic wax, such as the product sold under the name Micro Care 325® by the company Micro Powders, and polyethylene microwaxes, such as those sold under the names Micropoly 200®, 220®, 220L® and 250S® by the company Micro Powders.

The polymethylmethacrylate powder may be in the form of hollow or solid white spherical particles generally with a number-average size of micrometer order, for example, ranging from 3 to 15 microns and, further, for example, ranging from 3 to 10 microns. As used herein, the expression “number-average size” means the size given by the statistical particle size distribution to half of the population, referred to as D50.

It is also possible to characterize the polymethylmethacrylate particles by their density, which can vary, for example, as a function of the size of the spherical cavity of the particles.

For example, the density of the polymethylmethacrylate powder that may be used in the embodiments disclosed herein may range, for example, from 0.3 to 1.5, further, for example, from 0.5 to 1.5 and, even further, for example, from 1 to 1.5.

As non-limiting illustrations of the polymethylmethacrylate powder that is suitable for use in the composition disclosed herein, mention may be made, for example, of the polymethylmethacrylate particles sold by the company Matsumoto Yushi Co. under the name “Micropearl M100”, by the company LCW under the name “Covabead LH 85” and those sold by the company Nihon Junyaku under the name “Jurymer MB1”.

The polyacrylonitrile powder may be chosen from acrylonitrile homopolymer powders and acrylonitrile copolymer powders, and, for example, expanded hollow particles of acrylonitrile homopolymer or copolymer. For example, the powders may be made of any expanded acrylonitrile homopolymer or copolymer that is non-toxic and a non-irritant to the skin.

It is possible to use, for example, a copolymer comprising: from 0% to 60% of units derived from vinylidene chloride, from 20% to 90% of units derived from acrylonitrile and from 0% to 50% of units derived from an acrylic or styrene monomer, wherein the sum of the percentages (by weight) is equal to 100. The acrylic monomer may, for example, be a methyl or ethyl acrylate or methacrylate. The styrene monomer may, for example, be α-methylstyrene or styrene.

In one embodiment, the powders used in the composition disclosed herein are chosen from hollow particles of an expanded copolymer of vinylidene chloride and of acrylonitrile or of vinylidene chloride and of acrylonitrile and of methacrylate. These powders may be dry or hydrated.

The powders may be obtained, for example, according to the processes disclosed in Patent and Patent Application Nos. EP 56219, EP 348372, EP 486080, EP 320473, EP 112807 and U.S. Pat. No. 3,615,972.

The internal cavity of the powder particles in principle comprises at least one gas, which may be chosen from air, nitrogen, and hydrocarbons, such as isobutane and isopentane.

The powder particles may be chosen, for example, from expanded terpolymer micro-spheres of vinylidene chloride, of acrylonitrile and of methacrylate, sold under the brand name Expancel by the company Expancel under the references 551 DE 50 (particle size of 40 μm), 551 DE 20 (particle size of 30 μm and mass per unit volume of 65 kg/m³), 551 DE 12 (particle size of 12 μm), 551 DE 80 (particle size of 80 μm) and 461 DE 50 (particle size of 50 μm). It is also possible to use microspheres formed from the same expanded terpolymer having a particle size of 8 μm and a mass per unit volume of 70 kg/m³, referred to hereinbelow as EL 23, or having a particle size of 34 μm and a mass per unit volume of 20 kg/m³, referred to hereinbelow as EL 43.

The polyurethane powder may be a powder of a copolymer of hexamethylene diisocyanate and trimethylol hexyl lactone. Such a polyurethane powder is sold, for example, under the names “Plastic Powder D-400” and “Plastic Powder D-800” by the company Toshiki. Other polyurethane powders that may be used include the product sold under the name “Plastic Powder CS-400” by the company Toshiki.

The polyamide powders useful in the invention may be those listed under the CTFA name of “Nylon 12” or “Nylon 6”. A mixture of particles and, for example, a mixture of Nylon-6 and Nylon-12 may be used.

The polyamide powder particles used in the invention include those sold under the names “Orgasol” by the company Atochem. The process for obtaining these particles is, for example, the process described in Patent Application Publication No. FR-A-2 619 385 or No. EP-A-303 530. These polyamide powder particles are moreover known according to their various physicochemical properties under the name “polyamide 12” or “polyamide 6”.

The polyamide powder particles useful in the present invention may also include those sold under the name SP500 by the company TORAY

The organopolysiloxane may be elastomeric or non-elastomeric. It is preferable to use elastomeric organopolysiloxane powder or organopolysiloxane elastomer powder.

The elastomeric organopolysiloxane may, for example, be crosslinked and may be obtained

via a crosslinking addition reaction of diorganopolysiloxane comprising at least one hydrogen linked to silicon and of diorganopolysiloxane comprising at least one ethylenically unsaturated group linked to silicon, preferably, in the presence of, for example, a platinum catalyst; or
via a dehydrogenation crosslinking condensation reaction between a diorganopolysiloxane comprising at least one hydroxyl end group and a diorganopolysiloxane comprising at least one hydrogen linked to silicon, preferably, in the presence of, for example, an organotin compound; or
via a crosslinking condensation reaction of a diorganopolysiloxane comprising at least one hydroxyl end group and of a hydrolysable organopolysilane; or
via thermal crosslinking of organopolysiloxane, preferably, in the presence of, for example, an organoperoxide catalyst; or
via crosslinking of organopolysiloxane by high-energy radiation such as gamma rays, ultraviolet rays or an electron beam.

In one embodiment, the elastomeric organopolysiloxane powder is crosslinked and is obtained via a crosslinking addition reaction of a diorganopolysiloxane (B2) comprising at least two hydrogens, each linked to a silicon, and of a diorganopolysiloxane (A2) comprising at least two ethylenically unsaturated groups linked to silicon, preferably, in the presence of, for example, a platinum catalyst (C2), for instance as described in Patent Application Publication No. EP-A-295886.

For example, the organopolysiloxane may be obtained via a reaction of dimethylpolysiloxane comprising dimethylvinylsiloxy end groups and of methylhydrogenopolysiloxane comprising trimethylsiloxy end groups, in the presence of a platinum catalyst.

Compound (A2) is the base reagent for the formation of elastomeric organopolysiloxane and the crosslinking takes place via an addition reaction of compound (A2) with compound (B2) in the presence of the catalyst (C2).

Compound (A2) may, for example, be a diorganopolysiloxane comprising at least two lower alkenyl groups (for example C2-C4); the lower alkenyl group may be chosen from vinyl, allyl and propenyl groups. These lower alkenyl groups may be located in any position of the organopolysiloxane molecule, but in one embodiment are located at the ends of the organopolysiloxane molecule. The organopolysiloxane (A2) may have a branched-chain, linear-chain, cyclic or network structure; in one embodiment, the linear-chain structure may be used. Compound (A2) may have a viscosity ranging from the liquid state to the gum state. For example, compound (A2) may have a viscosity of at least 100 centistokes at 25° C.

The organopolysiloxanes (A2) may be chosen from methylvinylsiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethylsiloxane-diphenylsiloxane-methylvinylsiloxane copolymers comprising dimethylvinylsiloxy end groups, dimethyl-siloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxane comprising dimethylvinylsiloxy end groups, and dimethylsiloxane-methyl(3,3,3-trifluoropropyl)siloxane copolymers comprising dimethylvinylsiloxy end groups.

Compound (B2) may, for example, be an organopolysiloxane comprising at least two hydrogens linked to silicon in each molecule and is thus the crosslinking agent for the compound (A2).

In one embodiment, the sum of the number of ethylenic groups per molecule of compound (A2) and the number of hydrogen atoms linked to silicon per molecule of compound (B2) is at least 4.

Compound (B2) may be of any molecular structure. In one embodiment, compound (B2) is of linear-chain or branched-chain structure or cyclic structure.

Compound (B2) may have a viscosity at 25° C. ranging from 1 to 50,000 centistokes, for example, in order to have good miscibility with compound (A2).

In one embodiment, compound (B2) may be added in an amount such that the molecular ratio between the total amount of hydrogen atoms linked to silicon in compound (B2) and the total amount of all the ethylenically unsaturated groups in compound (A2) is within the range from 1:1 to 20:1.

Compound (B2) may be chosen from methylhydrogenopolysiloxanes comprising trimethylsiloxy end groups, dimethylsiloxane-methylhydrogenosiloxane copolymers comprising trimethylsiloxy end groups, and cyclic dimethylsiloxane-methylhydrogenosiloxane copolymers.

Compound (C2) is the crosslinking reaction catalyst, and may, for example, be chosen from chloroplatinic acid, chloroplatinic acid-olefin complexes, chloroplatinic acid-alkenylsiloxane complexes, chloroplatinic acid-diketone complexes, platinum black, and platinum on a support.

The catalyst (C2) may, for example, be added in an amount ranging from 0.1 to 1000 parts by weight and, further, for example, from 1 to 100 parts by weight, as clean platinum metal, per 1000 parts by weight of the total amount of compounds (A2) and (B2).

Other organic groups may be linked to silicon in the organopolysiloxanes (A2) and (B2) described previously, for example, alkyl groups, such as methyl, ethyl, propyl, butyl or octyl; substituted alkyl groups, such as 2-phenylethyl, 2-phenylpropyl or 3,3,3-tri-fluoropropyl; aryl groups, such as phenyl, tolyl or xylyl; substituted aryl groups, such as phenylethyl; and substituted monovalent hydrocarbon-based groups, such as an epoxy group, a carboxylate ester group or a mercapto group.

In some embodiments, the elastomeric organopolysiloxane powder may, for example, be chosen from non-emulsifying elastomers. As used herein, the term “non-emulsifying” means organopolysiloxane elastomers not comprising a hydrophilic chain, such as polyoxyalkylene or polyglycerolated units.

Spherical elastomeric organopolysiloxanes are, for example, described in Patent Application Publication Nos. JP-A-S61-194009, EP-A-242 219, EP-A-295 886 and EP-A-765 656, the contents of which are incorporated by reference.

Elastomer organopolysiloxane powders that may be used include those sold under the names “Dow Corning 9505 Powder” and “Dow Corning 9506 Powder” by the company Dow Corning. These powders have the INCI name: dimethicone/vinyl dimethicone crosspolymer.

The elastomeric organopolysiloxane powder may, for example, be chosen from elastomeric organopolysiloxane powders coated with silicone resin, for example, with silsesquioxane resin, as described, for example, in U.S. Pat. No. 5,538,793, the content of which is incorporated by reference. Such elastomeric powders are sold under the names “KSP-100”, “KSP-101”, “KSP-102”, “KSP-103”, “KSP-104” and “KSP-105” by the company Shin-Etsu, and have the INCI name: vinyl dimethicone/methicone silsesquioxane crosspolymer.

Other elastomeric organopolysiloxanes in the form of spherical powders may be powders of hybrid silicone functionalized with fluoroalkyl groups, sold, for example, under the name “KSP-200” by the company Shin-Etsu and powders of hybrid silicones functionalized with phenyl groups, sold, for example, under the name “KSP-300” by the company Shin-Etsu.

### Fibers

Among fibers that may be used, mention may be made of fibers of synthetic or natural, mineral or organic origin. They may be short or long, individual or organized, for example braided, and hollow or solid. They may have any shape and may especially have a circular or polygonal (square, hexagonal or octagonal) cross section depending on the specific application envisaged. In particular, their ends are blunted and/or polished to prevent injury. The fibers have a length ranging from 1 μm to 10 mm, preferably from 0.1 mm to 5 mm and more preferably from 0.3 mm to 3 mm. Their cross section may include in a circle with a diameter ranging from 2 nm to 500 μm, preferably ranging from 100 nm to 100 μm and more preferably from 1 μm to 50 μm. As fibers that can be used in the powdery cosmetic compositions according to the present invention, mention may be made of non-rigid fibers such as polyamide (Nylon®) fibers or rigid fibers such as polyimideamide fibers, for instance those sold under the names Kermel® and Kermel Tech® by the company Rhodia or poly(p-phenyleneterephthalamide) (or aramid) fibers sold especially under the name Kevlar® by the company DuPont de Nemours, and mixtures thereof.

The additional filler(s) may be present in the composition in a content of greater than or equal to 5% by weight relative to the weight of composition, for example ranging from 1% to 80% by weight, preferably from 3% to 75% by weight, and more preferably from 5% to 70% by weight, relative to the total weight of the composition.

Coloring Agent(s)

The pulverulent phase according to the present invention may comprise at least one coloring agent.

The term “coloring agents” should be understood as encompassing pigments, nacres, and reflective particles, and mixtures thereof. The coloring agent may be represented as dyestuff

### Pigments

The term “pigments” should be understood to mean white or colored, mineral or organic particles of any shape, which are insoluble in a physiological medium, and which are intended to color the composition. The pigments may be white or colored, and mineral and/or organic.

Among the mineral pigments that may be mentioned are titanium dioxide, such as pigmentary titanium dioxide rutile type, optionally surface-treated, zirconium oxide or cerium oxide, and also zinc oxide, iron (black, yellow or red) oxide or chromium oxide, manganese violet, ultramarine blue, chromium hydrate and ferric blue, and metal powders, for instance aluminium powder and copper powder.

The organic pigments may be chosen from the materials below, and mixtures thereof:
cochineal carmine,
organic pigments of azo dyes, anthraquinone dyes, indigoid dyes, xanthene dyes, pyrene dyes, quinoline dyes, triphenylmethane dyes and fluorane dyes.

Among the organic pigments, mention may be made especially of the D&C certified pigments known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, and FD&C Yellow No. 6.

The chemical materials corresponding to each of the organic dyestuffs mentioned previously are mentioned in the publication “International Cosmetic Ingredient Dictionary and Handbook”, 1997 edition, pages 371 to 386 and 524 to 528, published by The Cosmetic, Toiletries and Fragrance Association, the content of which is incorporated into the present patent application by reference.

### Nacres

The term “nacres” should be understood as meaning colored particles of any form, which may or may not be iridescent, especially produced by certain molluscs in their shell, or alternatively synthesized, and which have a color effect via optical interference.

Examples of nacres that may be mentioned include nacreous pigments such as titanium mica coated with an iron oxide, mica coated with bismuth oxychloride, titanium mica coated with chromium oxide, and nacreous pigments based on bismuth oxychloride. They may also be mica particles at the surface of which are superposed at least two successive layers of metal oxides and/or of organic dyestuffs. The nacres may more particularly have a yellow, pink, red, bronze, orange, brown, gold and/or coppery color or glint.

As illustrations of nacres that may be introduced into the composition, mention may be made of the gold-colored nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

As further examples of nacres, mention may also be made of particles comprising a borosilicate substrate coated with titanium oxide.

Particles having a glass substrate coated with titanium oxide are especially sold under the name Metashine MC1080RY by the company Toyal.

Finally, examples of nacres that may also be mentioned include polyethylene terephthalate flakes, especially those sold by the company Meadowbrook Inventions under the name Silver 1P 0.004×0.004 (silver flakes).

### Reflective Particles

The term “reflective particles” denotes particles whose size, structure, especially the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, points of overbrightness that are visible to the naked eye, i.e. more luminous points that contrast with their environment by appearing to sparkle.

The reflective particles may be selected so as not to significantly alter the coloration effect generated by the coloring agents with which they are combined, and more particularly so as to optimize this effect in terms of color yield. They may more particularly have a yellow, pink, red, bronze, orange, brown, gold and/or coppery color or tint.

These particles may have varied forms and may especially be in platelet or globular form, in particular in spherical form.

The reflective particles, whatever their form, may or may not have a multilayer structure and, in the case of a multilayer structure, may have, for example, at least one layer of uniform thickness, in particular of a reflective material.

When the reflective particles do not have a multilayer structure, they may be composed, for example, of metal oxides, especially titanium or iron oxides obtained synthetically.

When the reflective particles have a multilayer structure, they may comprise, for example, a natural or synthetic substrate, especially a synthetic substrate at least partially coated with at least one layer of a reflective material, especially of at least one metal or metallic material. The substrate may be made of one or more organic and/or inorganic materials.

More particularly, it may be chosen from glasses, ceramics, graphite, metal oxides, aluminas, silicas, silicates, especially aluminosilicates and borosilicates, and synthetic mica, and mixtures thereof, this list not being limiting.

The reflective material may comprise a layer of metal or of a metallic material.

Again as an example of reflective particles comprising a mineral substrate coated with a layer of metal, mention may also be made of particles comprising a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the name Crystal Star GF 550 and GF 2525 by this same company.

Use may also be made of particles comprising a metallic substrate such as silver, aluminium, iron, chromium, nickel, molybdenum, gold, copper, zinc, tin, manganese, steel, bronze or titanium, said substrate being coated with at least one layer of at least one metal oxide such as titanium oxide, aluminium oxide, iron oxide, cerium oxide, chromium oxide or silicon oxides, and mixtures thereof.

Examples that may be mentioned include aluminium powder, bronze powder or copper powder coated with SiO₂ sold under the name Visionaire by the company Eckart.

The colouring agent(s) may preferably be present in the composition in a content of greater than or equal to 1% by weight relative to the weight of composition, for example ranging from 0% to 30% by weight, preferably from 0.5% to 20% by weight and more preferably from 1% to 15% by weight, relative to the total weight of the composition.

### (II) Liquid Phase

The powdery cosmetic composition according to the present invention may comprise at least one liquid phase. This liquid phase may advantageously serve as binder for the said pulverulent phase. The liquid phase preferably comprises at least one non-volatile hydrocarbon-based oil and/or silicone oil, more preferably at least one non-volatile silicone oil, and still more preferably a combination of non-volatile silicone oils.

The term “liquid” refers to a composition that is liquid at room temperature (25° C.) and atmospheric pressure (760 mmHg).

The term “non-volatile oil” means an oil that remains on the skin or keratin fibers at room temperature and pressure. More precisely, a non-volatile oil has an evaporation rate strictly less than 0.01 mg/cm²/min.

The powdery cosmetic composition according to the present invention advantageously has a content of liquid phase, and in particular of non-volatile oil(s), and more particularly of non-volatile silicone oil(s), of 0.5% by weight or more, preferably 1% by weight or more, more preferably from 1.5% to 10% by weight and even more preferably from 2% to 8% by weight in relation to the total weight of the powdery cosmetic composition.

### Hydrocarbon-Based Non-Volatile Oil

A liquid phase may comprise at least one non-volatile hydrocarbon-based oil. A composition according to the present invention may comprise one or more non-volatile hydrocarbon-based oils.

Non-volatile hydrocarbon-based oils that may especially be mentioned include:
hydrocarbon-based oils of plant origin, such as phytostearyl esters, such as phytostearyl oleate, phytostearyl isostearate and lauroyl/octyldodecyl/phytostearyl glutamate; triglycerides formed from fatty acid esters of glycerol, in particular whose fatty acids may have chain lengths ranging from C18 to C36, these oils possibly being linear or branched, and saturated or unsaturated; these oils may especially be heptanoic or octanoic triglycerides, shea oil, alfalfa oil, poppy oil, pumpkin oil, millet oil, barley oil, quinoa oil, rye oil, candlenut oil, passionflower oil, shea butter oil, aloe oil, sweet almond oil, peach stone oil, groundnut oil, argan oil, avocado oil, baobab oil, borage oil, broccoli oil, calendula oil, camellina oil, carrot oil, safflower oil, hemp oil, rapeseed oil, cottonseed oil, coconut oil, marrow seed oil, wheatgerm oil, jojoba oil, lily oil, macadamia oil, corn oil, meadowfoam oil, St-John's wort oil, monoi oil, hazelnut oil, apricot kernel oil, walnut oil, olive oil, evening primrose oil, palm oil, blackcurrant pip oil, kiwi seed oil, grape seed oil, pistachio oil, pumpkin oil, quinoa oil, musk rose oil, sesame oil, soybean oil, sunflower oil, castor oil and watermelon oil, and mixtures thereof, or alternatively caprylic/capric acid triglycerides, such as those sold by the company Stearineries Dubois or those sold under the names Miglyol 810®, 812® and 818® by the company Dynamit Nobel;
synthetic ethers containing from 10 to 40 carbon atoms;
synthetic esters, for instance the oils of formula R1COOR2, in which R1 represents at least one linear or branched fatty acid residue comprising from 1 to 40 carbon atoms and R2 represents a hydrocarbon-based chain, which is especially branched, containing from 1 to 40 carbon atoms, with the proviso that R1+R2 is greater than or equal to 10; these esters may be chosen especially from fatty acid esters of alcohols, for instance cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoates, polyethylene glycol diheptanoate, propylene glycol 2-diethylhexanoate, and mixtures thereof, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate;
polyol esters and pentaerythritol esters, for instance dipentaerythrityl tetrahydroxystearate/tetraisostearate;
esters of diol dimers and of diacid dimers;
copolymers of diol dimer and of diacid dimer and esters thereof, such as dilinoleyl diol dimer/dilinoleic dimer copolymers, and esters thereof;
copolymers of polyols and of diacid dimers, and esters thereof;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
C₁₂-C₂₂ higher fatty acids, such as oleic acid, linoleic acid and linolenic acid, and mixtures thereof;
dialkyl carbonates, the two alkyl chains possibly being identical or different, such as dicaprylyl carbonate;
oils with a molar mass of between about 400 and about 10,000 g/mol, in particular about 650 to about 10,000 g/mol, more particularly from about 750 to about 7500 g/mol and even more particularly ranging from about 1000 to about 5000 g/mol; mention may be made especially of, alone or as a mixture, (i) lipophilic polymers such as polybutylenes, polyisobutylenes, for example hydrogenated, polydecenes and hydrogenated polydecenes, vinylpyrrolidone copolymers, such as the vinylpyrrolidone/1-hexadecene copolymer, and polyvinylpyrrolidone (PVP) copolymers, such as the copolymers of a C₂-C₃₀ alkene, such as C₃-C₂₂, and combinations thereof; (ii) linear fatty acid esters containing a total carbon number ranging from 35 to 70, for instance pentaerythrityl tetrapelargonate; (iii) hydroxylated esters such as polyglyceryl-2 triisostearate; (iv) aromatic esters such as tridecyl trimellitate; (v) esters of fatty alcohols or of branched C₂₄-C₂₈ fatty acids, such as those described in U.S. Pat. No. 6,491,927 and pentaerythritol esters, and especially triisoarachidyl citrate, pentaerythrityl tetraisononanoate, glyceryl triisostearate, glyceryl 2-tridecyltetradecanoate, pentaerythrityl tetraisostearate, poly(2-glyceryl) tetraisostearate or pentaerythrityl 2-tetradecyltetradecanoate; and (vi) diol dimer esters and polyesters, such as esters of diol dimer and of fatty acid, and esters of diol dimer and of diacid.

### Non-Volatile Silicone Oils

According to one preferred embodiment of the present invention, the liquid phase may comprise at least one non-volatile silicone oil. The non-volatile silicone oil that may be used in the present invention may be chosen from silicone oils with a viscosity at 25° C. of greater than or equal to 2 centistokes (cSt) (2×10⁻⁶ m²/s) and less than 800,000 cSt, preferably between 3 and 600,000 cSt and preferably between 4 and 500,000 cSt. The viscosity of this silicone may be measured according to standard ASTM D-445.

Among these silicone oils, two types of oil may be distinguished, according to whether or not they contain phenyl.

Representative examples of these non-volatile linear silicone oils that may be mentioned include polydimethylsiloxanes; alkyl dimethicones; vinyl methyl methicones; and also silicones modified with optionally fluorinated aliphatic groups, or with functional groups such as hydroxyl, thiol and/or amine groups.

Thus, non-phenyl non-volatile silicone oils that may be mentioned include:
PDMSs comprising alkyl or alkoxy groups, which are pendent and/or at the end of the silicone chain, these groups each containing from 2 to 24 carbon atoms,
PDMSs comprising aliphatic groups, or functional groups such as hydroxyl, thiol and/or amine groups,
polyalkylmethylsiloxanes optionally substituted with a fluorinated group, such as polymethyltrifluoropropyldimethylsiloxanes,
polyalkylmethylsiloxanes substituted with functional groups such as hydroxyl, thiol and/or amine groups,
polysiloxanes modified with fatty acids, fatty alcohols or polyoxyalkylenes, and mixtures thereof.

According to one particular embodiment, the powdery cosmetic composition according to the present invention contains at least one non-phenyl linear silicone oil. The non-phenyl linear silicone oil may be chosen especially from the silicones of formula:

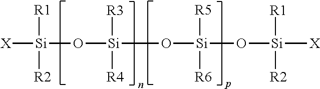

in which:
R₁, R₂, R₅ and R₆ are, together or separately, an alkyl radical containing 1 to 6 carbon atoms,
R₃ and R₄ are, together or separately, an alkyl radical containing from 1 to 6 carbon atoms, a vinyl radical, an amine radical or a hydroxyl radical,
X is an alkyl radical containing from 1 to 6 carbon atoms, a hydroxyl radical or an amine radical,
n and p are integers chosen so as to have a fluid compound.

As non-volatile silicone oils that may be used according to the invention, mention may be made of those in which:
the substituents R₁ to R₆ and X represent a methyl group, and p and n are such that the viscosity is 500,000 cSt, such as the product sold under the name SE30 by the company General Electric, the product sold under the name AK 500000 by the company Wacker, the product sold under the name Mirasil DM 500,000 by the company Bluestar, and the product sold under the name Dow Corning 200 Fluid 500,000 cSt by the company Dow Corning,
the substituents R₁ to R₆ and X represent a methyl group, and p and n are such that the viscosity is 60,000 cSt, such as the product sold under the name Dow Corning 200 Fluid 60000 CS by the company Dow Corning, and the product sold under the name Wacker Belsil DM 60,000 by the company Wacker,
the substituents R₁ to R₆ and X represent a methyl group, and p and n are such that the viscosity is 350 cSt, such as the product sold under the name Dow Corning 200 Fluid 350 CS by the company Dow Corning,
the substituents R₁ to R₆ represent a methyl group, the group X represents a hydroxyl group, and n and p are such that the viscosity is 700 cSt, such as the product sold under the name Baysilone Fluid T0.7 by the company Momentive.

According to one preferred embodiment variant, a composition according to the invention contains at least one phenyl silicone oil.

Representative examples of these non-volatile phenyl silicone oils that may be mentioned include:
The phenyl silicone oils corresponding to the following formula:

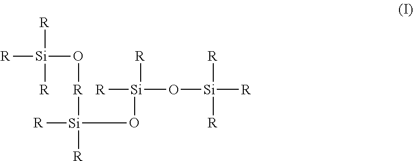

in which in formula (I) the groups R represent, independently of each other, a methyl or a phenyl, with the proviso that at least one group R represents a phenyl. Preferably, in this formula, the phenyl silicone oil comprises at least three phenyl groups, for example at least four, at least five or at least six.
The phenyl silicone oils corresponding to the following formula:

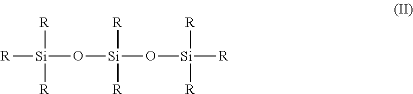

in which in formula (II) the groups R represent, independently of each other, a methyl or a phenyl, with the proviso that at least one group R represents a phenyl. Preferably, in this formula, the said organopolysiloxane comprises at least three phenyl groups, for example at least four or at least five. Mixtures of the phenyl organopolysiloxanes described previously may be used. Examples that may be mentioned include mixtures of triphenyl, tetraphenyl or pentaphenyl organopolysiloxanes.
The phenyl silicone oils corresponding to the following formula:

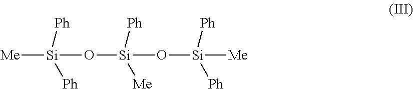 [SMILES: C[Si](O[Si](C)(c1ccccc1)c1ccccc1)(O[Si](C)(c1ccccc1)c1ccccc1)c1ccccc1]

in which in formula (III) Me represents methyl, and Ph represents phenyl. Such a phenyl silicone is especially manufactured by Dow Corning under the reference PH-1555 HRI or Dow Corning 555 Cosmetic Fluid (chemical name: 1,3,5-trimethyl-1,1,3,5,5-pentaphenyl trisiloxane; INCI name: trimethyl pentaphenyl trisiloxane). The reference Dow Corning 554 Cosmetic Fluid may also be used.
The phenyl silicone oils corresponding to the following formula:

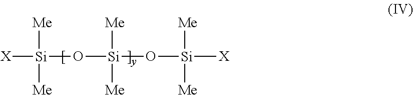

in which in formula (IV) Me represents methyl, y is between 1 and 1000 and X represents —CH₂—CH(CH₃)(Ph).
The phenyl silicone oils corresponding to formula (V) below:

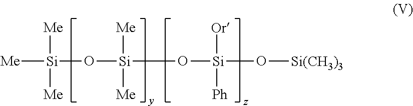

in which in formula (V) Me is methyl and Ph is phenyl, OR′ represents a group —OSiMe₃ and y is 0 or ranges between 1 and 1000, and z ranges between 1 and 1000, such that compound (V) is a non-volatile oil.

According to a first embodiment, y ranges between 1 and 1000. Use may be made, for example, of trimethyl siloxyphenyl dimethicone, especially under the reference Belsil PDM 1000 sold by the company Wacker.

According to a second embodiment, y is equal to 0. Use may be made, for example, of phenyl trimethylsiloxy trisiloxane, sold especially under the reference Dow Corning 556 Cosmetic Grade Fluid.
The phenyl silicone oils corresponding to formula (VI) below, and mixtures thereof:

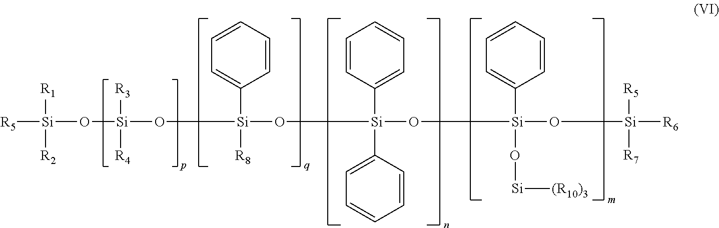

in which in formula (VI):
R₁ to R₁₀, independently of each other, are saturated or unsaturated, linear, cyclic or branched C₁-C₃₀ hydrocarbon-based radicals,
m, n, p and q are, independently of each other, integers between 0 and 900, with the proviso that the sum m+n+q is other than 0.

Preferably, the sum m+n+q is between 1 and 100. Preferably, the sum m+n+p+q is between 1 and 900 and more preferably between 1 and 800. Preferably, q is equal to 0.
The phenyl silicone oils corresponding to formula (VII) below, and mixtures thereof:

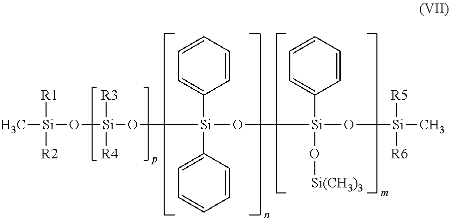

in which in formula (VII):
R₁ to R₆, independently of each other, are saturated or unsaturated, linear, cyclic or branched C₁-C₃₀ hydrocarbon-based radicals,
m, n and p are, independently of each other, integers between 0 and 100, with the proviso that the sum n+m is between 1 and 100.
   Preferably, R₁ to R₆, independently of each other, represent a saturated, linear or branched C₁-C₃₀ and especially C₁-C₁₂ hydrocarbon-based radical and in particular a methyl, ethyl, propyl or butyl radical.

R₁ to R₆ may especially be identical, and in addition may be a methyl radical. Preferably, m=1 or 2 or 3, and/or n=0 and/or p=0 or 1 may apply, in formula (VII).
The phenyl silicone oils corresponding to formula (VIII), and mixtures thereof:

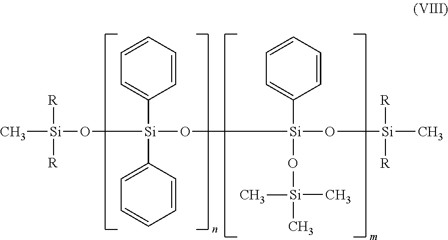

in which in formula (VIII):
R is a C₁-C₃₀ alkyl radical, an aryl radical or an aralkyl radical, preferably R is CH₃,
n is an integer ranging from 0 to 100, and
m is an integer ranging from 0 to 100, with the proviso that the sum n+m ranges from 1 to 100.
   In particular, the radicals R of formula (VIII) and R₁ to R₁₀ defined previously may each represent a linear or branched, saturated or unsaturated alkyl radical, especially of C₂-C₂₀, in particular C₃-C₁₆ and more particularly C₄-C₁₀, or a monocyclic or polycyclic C₆-C₁₄ and especially C₁₀-C₁₃ aryl radical, or an aralkyl radical whose aryl and alkyl residues are as defined previously.
   Preferably, R of formula (VIII) and R₁ to R₁₀ may each represent a methyl, ethyl, propyl, isopropyl, decyl, dodecyl or octadecyl radical, or alternatively a phenyl, tolyl, benzyl or phenethyl radical.
   According to one embodiment, a phenyl silicone oil of formula (VIII) with a viscosity at 25° C. of between 5 and 1500 mm²/s (i.e. 5 to 1500 cSt), and preferably with a viscosity of between 5 and 1000 mm²/s (i.e. 5 to 1000 cSt) may be used.
   As phenyl silicone oils of formula (VIII), it is especially possible to use phenyl trimethicones such as DC556 from Dow Corning (22.5 cSt), the oil Silbione 70663V30 from Rhône-Poulenc (28 cSt) or diphenyl dimethicones such as Belsil oils, especially Belsil PDM1000 (1000 cSt), Belsil PDM 200 (200 cSt) and Belsil PDM 20 (20 cSt) from Wacker. The values in parentheses represent the viscosities at 25° C.
The phenyl silicone oils corresponding to the following formula, and mixtures thereof:

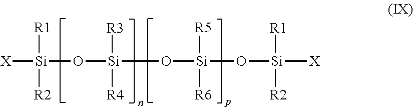

in which in formula (IX):
R₁, R₂, R₅ and R₆ are, together or separately, an alkyl radical containing 1 to 6 carbon atoms,
R₃ and R₄ are, together or separately, an alkyl radical containing from 1 to 6 carbon atoms or an aryl radical,
X is an alkyl radical containing from 1 to 6 carbon atoms, a hydroxyl radical or a vinyl radical,
n and p being chosen so as to give the oil a weight-average molecular mass of less than 200,000 g/mol, preferably less than 150,000 g/mol and more preferably less than 100,000 g/mol.

The phenyl silicones that are most particularly suitable for use in the invention are those corresponding to formulae (II) and especially to formulae (III), (V) and (VIII) above. More particularly, the phenyl silicones are chosen from phenyl trimethicones, phenyl dimethicones, diphenylsiloxy phenyl trimethicone, phenyl-trimethylsiloxydiphenylsiloxanes, diphenyl dimethicones, diphenylmethyldiphenyltrisiloxanes and 2-phenylethyl trimethylsiloxysilicates, and mixtures thereof.

Preferably, the weight-average molecular weight of the non-volatile phenyl silicone oil according to the invention ranges from 500 to 10,000 g/mol.

It should be noted that, among the silicone compounds according to the invention, phenyl silicone oils prove to be particularly advantageous.

### Volatile Oil

The liquid phase may optionally comprise at least one volatile oil. The term “volatile oil” means an oil (or non-aqueous medium) that can evaporate on contact with the skin in less than one hour, at room temperature and atmospheric pressure. The volatile oil is a cosmetic volatile oil, which is liquid at room temperature. More specifically, a volatile oil has an evaporation rate of between 0.01 and 200 mg/cm²/min, limits included.

To measure this evaporation rate, 15 g of oil or of oil mixture to be tested are placed in a crystallizing dish 7 cm in diameter, which is placed on a balance in a large chamber of about 0.3 m³ that is temperature-regulated, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing said oil or said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit of area (cm²) and per unit of time (minutes).

This volatile oil may be a hydrocarbon-based oil, silicone oil or fluoro oil. It is preferably a hydrocarbon-based oil.

The term “hydrocarbon-based oil” means an oil mainly containing hydrogen and carbon atoms.

The term “silicone oil” means an oil containing at least one silicon atom, and especially containing Si—O groups. According to one embodiment, the composition comprises less than 10% by weight of non-volatile silicone oil(s), in relation to the total weight of the powdery cosmetic composition, better still less than 5% by weight, or even is free of silicone oil.

The term “fluoro oil” means an oil comprising at least one fluorine atom.

The oils may optionally comprise oxygen, nitrogen, sulfur and/or phosphorus atoms, for example in the form of hydroxyl or acid radicals.

The volatile oils may be chosen from hydrocarbon-based oils containing from 8 to 16 carbon atoms, and especially C₈-C₁₆ branched alkanes (also known as isoparaffins), for instance isododecane, isodecane and isohexadecane.

The volatile hydrocarbon-based oil may also be a linear volatile alkane containing from 7 to 17 carbon atoms, in particular from 9 to 15 carbon atoms and more particularly from 11 to 13 carbon atoms. Mention may be made especially of n-nonadecane, n-decane, n-undecane, n-dodecane, n-tridecane, n-tetradecane, n-pentadecane and n-hexadecane, and mixtures thereof.

Preferably, the liquid phase is free of volatile oil. Such an absence of volatile oil makes it possible, where appropriate, to dispense with a perfectly leaktight conditioning assembly for the composition.

The liquid phase preferably comprises at least one non-volatile silicone oil, preferably at least one phenylated silicone oil and at least one non-phenylated silicone oil.

### Liquid UV Filter

The powdery cosmetic composition according to the present invention can comprise a further additional organic liquid UV filter other than the inorganic UV filter powder. If an organic liquid UV filter(s) is/are used in the liquid phase as the additional UV filter(s), the organic liquid UV filter(s) may be selected from the group consisting of anthranilic derivatives; dibenzoylmethane derivatives; liquid cinnamic derivatives; salicylic derivatives; camphor derivatives; benzophenone derivatives; β,β-diphenylacrylate derivatives; liquid triazine derivatives; liquid benzotriazole derivatives; benzalmalonate derivatives; benzimidazole derivatives; imidazoline derivatives; bis-benzoazolyl derivatives; p-aminobenzoic acid (PABA) and derivatives thereof; methylenebis(hydroxyphenylbenzotriazole) derivatives; benzoxazole derivatives; screening polymers and screening silicones; dimers derived from α-alkylstyrene; 4,4-diarylbutadienes; octocrylene and derivatives thereof, guaiazulene and derivatives thereof, rutin and derivatives thereof, flavonoids, biflavonoids, oryzanol and derivatives thereof, quinic acid and derivatives thereof, phenols, retinol, cysteine, aromatic amino acids, peptides having an aromatic amino acid residue, and mixtures thereof.

Mention may be made, as examples of the organic liquid UV filter(s), of those denoted below under their INCI names, and mixtures thereof.

Anthranilic derivatives: Menthyl anthranilate, marketed under the trademark “Neo Heliopan MA” by Haarmann and Reimer.

Dibenzoylmethane derivatives: Butyl methoxydibenzoylmethane, marketed in particular under the trademark “Parsol 1789” by Hoffmann-La Roche; and isopropyl dibenzoylmethane.

Liquid cinnamic derivatives: Ethylhexyl methoxycinnamate, marketed in particular under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate.

Salicylic derivatives: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer.

Camphor derivatives, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; terephthalylidene dicamphor sulfonic acid, manufactured under the trademark “Mexoryl SX” by Chimex; and polyacrylamidomethyl benzylidene camphor, manufactured under the trademark “Mexoryl SW” by Chimex.

Benzophenone derivatives: Benzophenone-1 (2,4-dihydroxybenzophenone), marketed under the trademark “Uvinul 400” by BASF; benzophenone-2 (Tetrahydroxybenzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norquay; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF; benzophenone-12, and n-hexyl 2-(4-diethylamino-2-hydroxybenzoyl)benzoate.

β,β-diphenylacrylate derivatives: Octocrylene, marketed in particular under the trademark “Uvinul N539” by BASF; and Etocrylene, marketed in particular under the trademark “Uvinul N35” by BASF.

Liquid triazine derivatives: diethylhexyl butamido triazone, marketed under the trademark “Uvasorb HEB” by Sigma 3V; 2,4,6-tris(dineopentyl 4′-aminobenzalmalonate)-s-triazine; and the symmetrical triazine screening agents described in U.S. Pat. No. 6,225,467, WO 2004/085412 (see Compounds 6 and 9) or the document “Symmetrical Triazine Derivatives”, IP.COM Journal, IP.COM INC, WEST HENRIETTA, N.Y., US (20 Sep. 2004), in particular 2,4,6-tris(biphenyl)-1,3,5-triazines (especially 2,4,6-tris(biphenyl-4-yl)-1,3,5-triazine) and 2,4,6-tris(terphenyl)-1,3,5-triazine, which are taken up again in WO 06/035000, WO 06/034982, WO 06/034991, WO 06/035007, WO 2006/034992 and WO 2006/034985.

Liquid benzotriazole derivatives, in particular, phenylbenzotriazole derivatives: 2-(2H-benzotriazole-2-yl)-6-dodecyl-4-methylpheno, branched and linear; and those described in U.S. Pat. No. 5,240,975.

Benzalmalonate derivatives: Dineopentyl 4′-methoxybenzalmalonate, and polyorganosiloxane comprising benzalmalonate functional groups, such as polysilicone-15, marketed under the trademark “Parsol SLX” by Hoffmann-LaRoche.

Benzimidazole derivatives, in particular, phenylbenzimidazole derivatives: Phenylbenzimidazole sulfonic acid, marketed in particular under the trademark “Eusolex 232” by Merck, and disodium phenyl dibenzimidazole tetrasulfonate, marketed under the trademark “Neo Heliopan AP” by Haarmann and Reimer.

Imidazoline derivatives: Ethylhexyl dimethoxybenzylidene dioxoimidazoline propionate.

Bis-benzoazolyl derivatives: The derivatives as described in Patent Application Publication No. EP-669,323 and U.S. Pat. No. 2,463,264.

Para-aminobenzoic acid and derivatives thereof: PABA (p-aminobenzoic acid), ethyl PABA, Ethyl dihydroxypropyl PABA, pentyl dimethyl PABA, ethylhexyl dimethyl PABA, marketed in particular under the trademark “Escalol 507” by ISP, glyceryl PABA, and PEG-25 PABA, marketed under the trademark “Uvinul P25” by BASF.

Methylenebis(hydroxyphenylbenzotriazole) derivatives: Methylene bis-benzotriazolyl tetramethylbutylphenol, marketed in the solid form under the trademark “Mixxim BB/100” by Fairmount Chemical or in the micronized form in aqueous dispersion under the trademark “Tinosorb M” by Ciba Specialty Chemicals, and the derivatives as described in U.S. Pat. Nos. 5,237,071 and 5,166,355, and Patent Application Publication GB-2,303,549, DE-197,26,184 and EP-893,119.

Benzoxazole Derivatives:

2,4-bis[5-1 (dimethylpropyl)benzoxazol-2-yl-(4-phenyl)imino]-6-(2-ethylhexyl)imino-1,3,5-triazi ne, marketed under the trademark of Uvasorb K2A by Sigma 3V.

Screening polymers and screening silicones: The silicones described in WO 93/04665.

Dimers derived from α-alkylstyrene: The dimers described in DE-19855649.

4,4-Diarylbutadiene Derivatives:

1,1-dicarboxy(2,2′-dimethylpropyl)-4,4-diphenylbutadiene.

Octocrylene and derivatives thereof: Octocrylene.

Quaiazulene and derivatives thereof: Guaiazulene and sodium guaiazulene sulfonate.

Rutin and derivatives thereof: Rutin and glucosylrutin.

Flavonoids: Robustin (isoflavonoid), genistein (flavonoid), tectochrysin (flavonoid), and hispidone (flavonoid).

Biflavonoids: Lanceolatin A, lanceolatin B, and hypnumbiflavonoid A.

Oryzanol and derivatives thereof: F-oryzanol.

Quinic acid and derivatives thereof: Quinic acid.

Phenols: Phenol.

Retinols: Retinol.

Cysteines: L-cysteine.

Peptides having an aromatic amino acid residue: Peptides having tryptophan, tyrosine or phenylalanine.

The preferred organic liquid UV filter(s) is selected from:

butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4,6-tris(biphenyl-4-yl)-1,3,5-triazine, 2,4,6-tris(terphenyl)-1,3,5-triazine, methylene bis-benzotriazolyl tetramethylbutylphenol, polysilicone-15, dineopentyl 4′-methoxybenzalmalonate, 1,1-dicarboxy(2,2′-dimethylpropyl)-4,4-diphenylbutadiene, 2,4-bis[5-1 (dimethylpropyl)benzoxazol-2-yl-(4-phenyl)imino]-6-(2-ethylhexyl)imino-1,3,5-triazine, and their mixtures.

The preferred liquid UV filter(s) is selected from the group consisting of ethylhexyl methoxycinnamate, ethylhexyl dimethyl PABA (p-aminobenzoic acid), ethylhexyl salicylate, octocrylene, and homosalate.

The liquid UV filter may be used in the composition according to the present invention in proportions such that the weight ratio of the inorganic UV filter powder to the liquid UV filter is 50:50 to 90:10, preferably 50:50 to 85:15, and more preferably 50:50 to 80:20.

### Additives

The powdery cosmetic composition according to the present invention may comprise other additives usually used in cosmetics, such as antioxidants, fragrances, preservatives, bactericides, neutralizing agents, surfactants, waxes, sunscreens, vitamins, moisturizing agents, self-tanning compounds or antiwrinkle active principles.

Examples of the antioxidants that may be used include BHT and tocopherol.

Examples of the preservatives that may be used include esters of para-hydroxybenzoic acid, also known as Parabens® (in particular methylparaben, ethylparaben or propylparaben), phenoxyethanol, releasers of formaldehyde, such as, for example, imidazolidinyl urea or diazolidinyl urea, chlorhexidine digluconate, sodium benzoate, caprylyl glycol, iodopropynyl butylcarbamate, pentylene glycol, alkyltrimethylammonium bromide, such as myristyltrimethylammonium bromide (CTFA name: myrtrimonium bromide), dodecyltrimethylammonium bromide, hexadecyltrimethylammonium bromide and their mixtures, such as the mixture marketed under the trademark Cetrimide® by FEF Chemicals. The preservative can be present in the composition according to the invention in a content ranging from 0.001% to 10% by weight, with respect to the total weight of the composition, in particular ranging from 0.1% to 5% by weight and especially ranging from 0.2% to 3% by weight.

Examples of the bactericides that may be used include a glyceryl mono(C₃-C₉)alkyl or mono(C₃-C₉)alkenyl ether, the manufacture of which is described in the literature, in particular in E. Baer, H. O. L. Fischer—J. Biol. Chem. 140-397-1941. Among these glyceryl mono(C₃-C₉)alkyl or mono(C₃-C₉)alkenyl ethers, 3-[(2-ethylhexyl)oxy]-1,2-propanediol, 3-[(heptyl)oxy]-1,2-propanediol, 3-[(octyl)oxy]-1,2-propanediol and 3-[(allyl)oxy]-1,2-propanediol are preferably used. A glyceryl mono(C₃-C₉)alkyl ether that is more particularly preferred according to the present invention is 3-[(2-ethylhexyl)oxy]-1,2-propanediol, sold by the company Schulke & Mayr GmbH under the trade name Sensiva SC 50 (INCI name: ethylhexylglycerin).

Examples of the surfactants that may be used include sucrose distearate, diglyceryl distearate, tetraglyceryl tristearate, decaglyceryl decastearate, diglyceryl monostearate, hexaglyceryl tristearate, decaglyceryl pentastearate, sorbitan monostearate, sorbitan tristearate, diethylene glycol monostearate, the glyceryl ester of palmitic or stearic acid, polyoxyethylene 2EO monosterate (comprising 2 oxyethylene units), glyceryl mono- and dibehenate, pentaerythrityl tetrastearate, polyoxyethylenated sorbitan monostearate 4EO, polyoxyethylenated sorbitan tristearate 20EO, polyoxyethylenated monostearate 8EO, hexaglyceryl monostearate, polyoxyethylenated monostearate 10EO, polyoxyethylenated distearate 12EO, polyoxyethylenated methylglucose distearate 20EO, and copolyol dimethicone such as cethyl PEG17 dimethicone.

Needless to say, a person skilled in the art will take care to select the optional additive(s) added to the composition such that the advantageous properties intrinsically associated with the cosmetic composition in accordance with the present invention are not, or are not substantially, adversely affected by the envisaged addition.

### [Preparation]

A manufacturing process of a powdery cosmetic composition according to the present invention comprises a step of (i) mixing the perlite and at least one inorganic UV filter powder having an average primary particle size of lower than 200 nm, preferably from 5 nm to 150 nm, and more preferably from 10 nm to 100 nm, to provide a pulverulent mixture, wherein an amount of the perlite is from 5 wt % to 70 wt % in relation to the total weight of the composition.

A manufacturing process according to the present invention may further comprise, after the step (i), a step of (ii) adding a liquid to the pulverulent mixture to provide a bulk mixture, (iii) pulverizing the bulk mixture, and optionally (iv) pressing the pulverized bulk mixture.

The step (iii) of pulverizing the bulk mixture is usually performed by well-known techniques in the art, for example, by using a mill, such as a hammer mill.

The powdery cosmetic composition according to the present invention can be provided in the form of a compact powder. If the above pressing step (iv) is not necessary, the powdery cosmetic composition according to the present invention can be provided in the form of a loose powder.

The optional step of (iv) pressing the pulverized bulk mixture is performed by applying a pressure ranging from 0.5 MPa to 10 MPa. In one embodiment of the present invention, the pulverized bulk mixture may be pressed by applying a pressure ranging from 1 MPa to 5 MPa.

The powdery cosmetic composition according to the present invention can be used as various powdery cosmetic products, such as make-up products, in particular powdery foundations.

The manufacturing process according to the present invention does not require any special industrial processes, for example special mixing or milling processes, which are expensive and complicated. This is because the powdery cosmetic composition of the present invention can achieve good UV protecting effects without a use of a large amount of the inorganic UV filter powder.

### [Cosmetic Process]

In another aspect, the present invention also relates to a cosmetic process including the step of applying to skin, preferably the face, the powdery cosmetic composition according to the present invention. The cosmetic process preferably includes making up and/or caring for the skin, preferably facial skin.

In the case that the composition is in the form of a compact powder, the powder can be picked up with an applicator, such as a sponge, puff, or brush, by rubbing off the powder. Then the powder is moved from the applicator to the skin by contacting the applicator on the skin.

The powdery cosmetic composition used in the cosmetic process according to the present invention is preferably of the leave-in type. The term “leave-in” means a composition that is not intended to be washed out or removed immediately after application.

The cosmetic process according to the present invention can provide long lasting cosmetic effects, such as long lasting matte effects and/or color-keeping effects, as well as good UV protecting effects. Therefore, for example, skin imperfections, such as redness, marks, pores and wrinkles on the skin, in particular the face, can be masked for a long period of time. Thus, the cosmetic process according to the present invention can produce good staying power on the skin over time even under hot and/or humid conditions, for example, during summer.

Furthermore, the cosmetic process according to the present invention or the powdery cosmetic composition according to the present invention can also provide good feeling upon use, texture, spreadability, sebum resistance, sweat resistance and the like.

### EXAMPLES

The present invention will be described in a more detailed manner by way of examples. However, these examples should not be construed as limiting the scope of the present invention.

### Example 1 and Reference Examples 1 and 2

### Preparations

The following powdery cosmetic compositions according to Example (Ex.) 1 and Reference Examples (Ref.) 1 and 2, shown in Table 1, were prepared by mixing the components shown in Table 1. The numerical values for the amounts of the components shown in Table 1 are all based on “% by weight” as active raw materials. Also, the value in parenthese of the inorganic UV filter powder in Table 1 indicates a number-average size mean diameter (D50) measured with Mastersizer 2000 by Malvem Corp. As the titanium dioxide, the product “MPT-141” from Ishihara Sangyo was used.

The details of the components are shown below:
Perlite: average particle size; 10 μm
Talc A: average particle size; 25 μm
Talc B: average particle size; 10 μm
Mica: average particle size; 25 μm

TABLE 1

| Ingredient | Ex. 1 | Ref. 1 | Ref. 2 |
|---|---|---|---|
| Titanium Dioxide | 21.00 | 21.00 | 21.00 |
| (Inorganic UV Filter Powder, 100 nm) | | | |
| Perlite | 65.00 | — | — |
| Talc A | — | 65.00 | — |
| Talc B | — | — | 65.00 |
| Mica | 5.00 | 5.00 | 5.00 |
| Starch | 5.00 | 5.00 | 5.00 |
| Pulverulent phase | 96.00 | 96.00 | 96.00 |
| (Ethylhexyl methoxycinnamate) | 4.00 | 4.00 | 4.00 |
| (Liquid phase) | | | |
| Total | 100 | 100 | 100 |

### [UV Transmittance Evaluation]

The powdery cosmetic compositions (foundations) according to Example 1 and Reference Examples 1 and 2 were compared in terms of UV transmittance. UV transmittance was measured in accordance with following protocol:

(1) 5 wt % of the composition was dispersed in dimethicone (SHINETSU Silicone, KF96-12,500 centi-stokes) to form a suspension,

(2) the obtained suspension was held between two square quartz glasses (GL Science, catalogue#6220-71031, 30 mm*30 mm*1 mmt), and the light path length was controlled to 50 microns by means of a lead spacer (GL Science, catalogue#6220-20105, DC-You-Space, length: 0.05 mm), and then,

(3) transmittance was measured by means of a UV/Vis spectrophotometer (JASCO, UV-550) attached to an integrating sphere (JASCO, ISV-469).

At least three differently assembled samples were measured to confirm reproducibility. The results are shown in Table 2.

TABLE 2

| | Ex. 1 | Ref. 1 | Ref. 2 |
|---|---|---|---|
| UV Transmittance | 2.4 | 4.7 | 3.1 |
| (%) at (290) nm | | | |
| UV Transmittance | 14.4 | 35.3 | 21.9 |
| (%) at (340) nm | | | |

### Examples 2 and 3 and Reference Example 3

### Preparations

The following powdery cosmetic compositions according to Examples (Ex.) 2 and 3, and Reference Example (Ref.) 3, shown in Table 3, are prepared using the components shown in Table 3 in accordance with the following protocol. The numerical values for the amounts of the components shown in Table 3 are all based on “% by weight” as active raw materials. Also, the value in parenthese of the inorganic UV filter powder in Table 3 indicates a number-average size mean diameter (D50) measured by SEM (Scanning Electron Microscope) and/or TEM (Transmission Electron Microscope). The perlite used in these examples is the same as that used in Example 1 above.

### [Preparation Protocol]

(1) the entire amount of the powder components are mixed in a mixer to produce a pulveruent mixture,

(2) the liquid components are put into the pulveruent mixture to produce a bulk mixture,

(3) the bulk mixture is pulverized with a hammer mill to form a pulverized bulk mixture, and then

(4) the pulverized bulk mixture is pressed to form a powdery cosmetic composition (foundation).

TABLE 3

| Ingredient | Ex. 2 | Ex. 3 | Ref. 3 |
|---|---|---|---|
| Perlite | 10 | 40 | 0.1 |
| Titanium Dioxide with surface treatment | 12 | 6.5 | 12 |
| (Inorganic UV Filter Powder, 15 nm) | | | |
| Mica with surface tresatment | 7.5 | 7.5 | 7.5 |
| Talc | QS | QS | QS |
| Silica | 0.5 | 0.5 | 0.5 |
| Pigmentary Titanium Dioxide Rutile Type | 9 | 9 | 9 |
| Iron Oxide | 5 | 5 | 5 |
| Pulverulent phase | 92.4 | 92.4 | 92.4 |
| Ethylhexyl methoxycinnamate | 4 | 4 | 4 |
| Dimethicone | 3 | 3 | 3 |
| Ethylhexyl Glycerin | 0.2 | 0.2 | 0.2 |
| Caprylyl Glycol | 0.4 | 0.4 | 0.4 |
| Total | 100 | 100 | 100 |

### [Lastingness Evaluation]

The powdery cosmetic compositions (foundations) according to Examples 2 and 3, and Reference Example 3 are compared in terms of long lasting cosmetic effects (long lasting, hiding pores and lines, and coverage) for 5 days by panelists who applies 1 g of a sample to the face at once a day in Indonesia. The lastingness is evaluated by visual observation to each of panelists.

### [SPF Value Evaluation]

In vivo SPF value of the compositions according to Examples 2 and 3 is measured according to ISO-24444 method with an SPF analyzer UV-2000S. The results of this test are shown in Table 4.

TABLE 4

| | Lastingness | SPF Value |
|---|---|---|
| Ex. 2 | 5 to 7 hours | 21.2 |
| Ex. 3 | 7 to 10 hours | 25.4 |
| Ref. 3 | 1 to 3 hours | 19.0 |

The powdery cosmetic compositions according to Examples 2 and 3 can provide long lasting cosmetic effects, as well as UV protecting effects.

1.-11. (canceled)

12. A powdery cosmetic composition comprising a pulverulent phase, wherein the pulverulent phase comprises:
(i) perlite in an amount ranging from about 5 wt % to about 70 wt % relative to the total weight of the composition, and
(ii) at least one inorganic UV filter powder having an average primary particle size of less than about 200 nm.

13. The powdery cosmetic composition according to claim 12, wherein the particle size of perlite ranges from about 1 μm to about 50 μm.

14. The powdery cosmetic composition according to claim 12, wherein the particle size of the at least one inorganic UV filter powder ranges from about 10 nm to about 100 nm.

15. The powdery cosmetic composition according to claim 12, wherein the at least one inorganic UV filter powder is chosen from titanium dioxide, zinc oxide, cerium oxide, or mixtures thereof.

16. The powdery cosmetic composition according to claim 12, wherein the amount of the at least one inorganic UV filter powder ranges from about 3 wt % to about 40 wt % relative to the total weight of the composition.

17. The powdery cosmetic composition according to claim 12, wherein the composition is in the form of a compacted powder or a loose powder.

18. The powdery cosmetic composition according to claim 12, further comprising a liquid phase.

19. The powdery cosmetic composition according to claim 18, wherein the liquid phase comprises at least one liquid UV filter.

20. The powdery cosmetic composition according to claim 19, wherein the at least one liquid UV filter is chosen from ethylhexyl methoxycinnamate, ethylhexyl dimethyl PABA (p-aminobenzoic acid), ethylhexyl salicylate, octocrylene, homosalate, or mixtures thereof.

21. A method for making up and/or caring for the skin, the method comprising applying to the skin a powdery cosmetic composition comprising a pulverulent phase, wherein the pulverulent phase comprises:
(i) perlite in an amount ranging from about 5 wt % to about 70 wt % relative to the total weight of the composition, and
(ii) at least one inorganic UV filter powder having an average primary particle size of less than about 200 nm.

22. The method for making up and/or caring for the skin according to claim 21, wherein the particle size of perlite ranges from about 1 μm to about 50 μm.

23. The method for making up and/or caring for the skin according to claim 21, wherein the particle size of the at least one inorganic UV filter powder ranges from about 10 nm to about 100 nm.

24. The method for making up and/or caring for the skin according to claim 21, wherein the at least one inorganic UV filter powder is chosen from titanium dioxide, zinc oxide, cerium oxide, or mixtures thereof.

25. The method for making up and/or caring for the skin according to claim 21, wherein the amount of the at least one inorganic UV filter powder ranges from about 3 wt % to about 40 wt % relative to the total weight of the composition.

26. The method for making up and/or caring for the skin according to claim 21, wherein the composition is in the form of a compacted powder or a loose powder.

27. The method for making up and/or caring for the skin according to claim 21, further comprising a liquid phase.

28. The method for making up and/or caring for the skin according to claim 27, wherein the liquid phase comprises at least one liquid UV filter.

29. The method for making up and/or caring for the skin according to claim 28, wherein the at least one liquid UV filter is chosen from ethylhexyl methoxycinnamate, ethylhexyl dimethyl PABA (p-aminobenzoic acid), ethylhexyl salicylate, octocrylene, homosalate, or mixtures thereof.

30. A method for manufacturing a powdery cosmetic composition, said method comprising (i) mixing perlite and at least one inorganic UV filter powder to provide a pulverulent mixture, wherein the amount of perlite ranges from about 5 wt % to about 70 wt % relative to the total weight of the composition, and wherein the inorganic UV filter powder has an average primary particle size of less than about 200 nm.

31. The manufacturing method according to claim 30, further comprising: (ii) adding a liquid to the pulverulent mixture to provide a bulk mixture, (iii) pulverizing the bulk mixture, and optionally (iv) pressing the pulverized bulk mixture.
